(19) United States
(12) Patent Application Publication
(10) Pub. No.: US 2023/0363441 A1
(43) Pub. Date: Nov. 16, 2023

(54) FORMULATION

(71) Applicants: Nicoventures Trading Limited, LONDON (GB); RAI Strategic Holdings, Inc., Winston-Salem, NC (US)

(72) Inventors: Michael Foster DAVIS, Winston Salem, NC (US); Savannah JOHNSON, Winston-Salem, NC (US); Rei KAWAMURA, Winston-Salem, NC (US); Alice HUGHES, London (GB); Karina MCQUILLAN, London (GB); Nickolai SOLECHNIK, London (GB)

(21) Appl. No.: 18/246,606

(22) PCT Filed: Sep. 24, 2021
(86) PCT No.: PCT/GB2021/052481

§ 371 Date: Mar. 24, 2023

Related U.S. Application Data
(60) Provisional application No. 63/198,025, filed on Sep. 24, 2020.
(60) Provisional application No. 63/201,138, filed on Apr. 14, 2021.

(51) Int. Cl. A24B 15/40; A24B 15/30; A24B 15/167

(52) U.S. Cl. CPC A24B 15/40; A24B 15/303; A24B 15/167

(57) ABSTRACT

A formulation, a method of making said formulation, as well as containers and systems including and using said formulation. In particular, the formulation includes one or more cannabinoids, one or more stabilizing component and one or more carrier constituents, wherein the total amount of the one or more carrier constituents is 50% w/w or more based on the total weight of the formulation, wherein said formulation has a pH of less than about 7.5.

### PRIORITY CLAIM

The present application is a National Phase entry of PCT Application No. PCT/GB2021/052481, filed Sep. 24, 2021, which claims priority from U.S. Provisional Application No. 63/198,025, filed Sep. 24, 2020 and U.S. Provisional Application No. 63/201,138, filed Apr. 14, 2021, each of which hereby fully incorporated herein by reference

### FIELD

The present disclosure relates to a formulation, a method of making said formulation, as well as containers and systems comprising and using said formulation.

### BACKGROUND

Aerosol delivery systems which generate an aerosol for inhalation by a user are known in the art. Such systems typically comprise an aerosol generator which is capable of converting a formulation into an aerosol. In some instances, the aerosol generated is a condensation aerosol whereby a formulation is heated to form a vapor which is then allowed to condense into an aerosol. In other instances, the aerosol generated is an aerosol which results from the atomization of the formulation. Such atomization may be brought about mechanically, e.g. by subjecting the formulation to vibrations so as to form small particles of material that are entrained in airflow. Alternatively, such atomization may be brought about electrostatically, or in other ways, such as by using pressure etc.

Depending on the constituents of the formulation that are to be provided to a user, it may be preferable to formulate the formulation in a certain way. For example, it may be preferable to formulate the formulation so as to produce an aerosol with a particular profile. It may also be preferable to formulate the formulation so as to ensure the formulation meets certain standards of quality, consistency and the like.

It would thus be desirable to provide a formulation that is formulated so as to be acceptable to a user.

### SUMMARY

In one aspect there is provided a formulation comprising one or more cannabinoids, one or more stabilizing components and one or more carrier constituents, wherein the total amount of the one or more carrier constituent is 50% w/w or more based on the total weight of the formulation, wherein said formulation has a pH of less than about 7.5.

In a further aspect there is provided a packaged formulation comprising a formulation as defined herein, wherein the packaged formulation is impermeable to air.

In a further aspect there is provided a method of producing a formulation as defined herein, wherein the method comprises combining each of the one or more cannabinoids, one or more stabilizing components and one or more carrier constituents so as to form the formulation, wherein the one or more stabilizing components and one or more carrier constituents are combined to form a first mixture, and then the one or more cannabinoids is added to the first mixture to produce the formulation.

In a further aspect there is provided a method of preparing a packaged formulation, wherein the method comprises packaging a formulation as defined herein, wherein the resulting container including the formulation comprises a volume of gas no greater than 20% of the total volume of the container.

In a further aspect there is provided a container comprising a formulation as defined herein, wherein the container including the formulation comprises a volume of gas no greater than 20% of the total volume of the container.

In a further aspect there is provided an article comprising the formulation as defined herein.

In a further aspect there is provided an aerosol provision system comprising an aerosol provision device and an article as defined herein.

These aspects and other aspects will be apparent from the following detailed description. In this regard, particular sections of the description are not to be read in isolation from other sections.

### BRIEF DESCRIPTION OF THE DRAWINGS

Various embodiments will now be described in detail by way of example only with reference to the accompanying drawings in which:

FIG. 1—Provides a solubility graph for a ternary system of propylene glycol/glycerol/cannabidiol

FIG. 2—Provides a schematic overview of an article, aerosol delivery device and system as described herein

### DETAILED DESCRIPTION

Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), Tetrahydrocannabinol (THC), including its isomers Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

Although the legal status of specific cannabinoids varies from jurisdiction to jurisdiction, certain active components, for example cannabidiol (CBD), tetrahydrocannabinol (THC) and cannabinol (CBN), are being considered for use in a wide variety of applications, such as in formulations for use in aerosol delivery systems. However, the stability of cannabinoids, such as cannabidiol (CBD), tetrahydrocannabinol (THC) and cannabinol (CBN), has been found to vary depending on certain environmental conditions, such as exposure to air or light, or variation in temperature and pH. This may have unintended and detrimental consequences. For example, CBD may oxidise and degrade when exposed to light and/or air to form cannabidiol hydroxyquinone (CBDHQ or HU-331) and its isomeric or functional derivatives. Furthermore, CBD may be converted to Δ⁹-tetrahydrocannabinol (Δ⁹-THC) in response to variations in temperature and/or pH. As a result, the accuracy of the specified cannabinoid content and/or concentration may vary widely in the formulations, while regulated and restricted cannabinoids may be produced unintentionally that will render the product as illicit or unlicensed in certain jurisdictions. As such, there is a desire to provide formulations comprising one or more cannabinoids that maintain a high degree of purity during manufacture and storage, and in turn prevent the loss or degradation of one or more cannabinoids, such as cannabidiol (CBD), tetrahydrocannabinol (THC) or cannabinol (CBN), in a formulation.

It has been found that by including one or more stabilizing components within a cannabinoid containing formulation, whilst controlling the pH of the formulation, it is possible to mitigate the extent to which derivatives of the cannabinoid of interest are formed. Without wishing to be bound by theory, it has been found that the use of antioxidants, radical scavengers, pH modulators, chelating agents and combinations thereof can be used as stabilizing components to attenuate the oxidation and/or degradation of cannabinoids, for example the formation of CBDHQ or Δ⁹-THC from CBD.

In one embodiment, the cannabinoid is a synthetic cannabinoid. In one embodiment, the cannabinoid is added to the formulation in the form of an isolate. An isolate is an extract from a plant, such as a cannabis plant. The cannabinoid(s) of interest are typically present in a high degree of purity, for example greater than 95%, greater than 96%, greater than 97%, greater than 98%, or around 99% purity. A synthetic cannabinoid is one which has been derived from a chemical synthesis as opposed to being isolated from a plant or biological source. In one embodiment the cannabinoid(s) of interest are selected from cannabigerol (CBG), cannabichromene (CBC), cannabidiol (CBD), Tetrahydrocannabinol (THC), including its isomers Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A). In one embodiment the cannabinoid(s) of interest are selected from cannabigerol (CBG), cannabichromene (CBC), cannabidiol (CBD), Δ⁸-tetrahydrocannabinol (Δ⁸-THC), Δ⁹-tetrahydrocannabinol (Δ⁹-THC) and cannabinol (CBN).

In one embodiment the cannabinoid(s) of interest are selected from cannabidiol (CBD), Δ⁸-tetrahydrocannabinol (Δ⁸-THC), Δ⁹-tetrahydrocannabinol (Δ⁹-THC).

In one embodiment the cannabinoid of interest is cannabidiol (CBD).

In one embodiment the cannabinoid of interest is Δ⁸-tetrahydrocannabinol (Δ⁸-THC).

In one embodiment the cannabinoid of interest is Δ⁹-tetrahydrocannabinol (Δ⁹-THC).

In one embodiment the cannabinoid of interest is cannabinol (CBN).

In one embodiment, the cannabinoid is cannabidiol (CBD) or a pharmaceutically acceptable salt thereof. In one embodiment, the cannabidiol (CBD) is synthetic cannabidiol (CBD). In one embodiment, the cannabidiol (CBD) is added to the formulation in the form of a cannabinoid isolate. In one embodiment the cannabinoid isolate comprises cannabidiol (CBD), wherein the cannabidiol (CBD) is present in a purity greater than 95%, greater than 96%, greater than 97%, greater than 98%, or around 99% purity.

The cannabinoid may be present in the formulation based on a mg/ml basis of the formulation.

In one embodiment, the cannabinoid is present in an amount of from about 5 mg/ml up to about 300 mg/ml. In one embodiment, the cannabinoid is present in an amount of from about 5 mg/ml up to about 250 mg/ml. In one embodiment, the cannabinoid is present in an amount of from about 5 mg/ml up to about 200 mg/ml. In one embodiment, the cannabinoid is present in an amount of from about 5 mg/ml up to about 150 mg/ml. In one embodiment, the cannabinoid is present in an amount of from about 5 mg/ml up to about 100 mg/ml. In one embodiment, the cannabinoid is present in an amount of from about 5 mg/ml up to about 90 mg/ml. In one embodiment, the cannabinoid is present in an amount of from about 5 mg/ml up to about 80 mg/ml. In one embodiment, the cannabinoid is present in an amount of from about 5 mg/ml up to about 70 mg/ml. In one embodiment, the cannabinoid is present in an amount of from about 5 mg/ml up to about 60 mg/ml. In one embodiment, the cannabinoid is present in an amount of from about 5 mg/ml up to about 50 mg/ml. In one embodiment, the cannabinoid is present in an amount of from about 5 mg/ml up to about 40 mg/ml. In one embodiment, the cannabinoid is present in an amount of from about 5 mg/ml up to about 30 mg/ml. In one embodiment, the cannabinoid is present in an amount of from about 5 mg/ml up to about 20 mg/ml. In one embodiment, the cannabinoid is present in an amount of from about 5 mg/ml up to about 10 mg/ml.

In one embodiment, the cannabinoid is present in an amount of about 5 mg/ml or more. In one embodiment, the cannabinoid is present in an amount of about 10 mg/ml or more. In one embodiment, the cannabinoid is present in an amount of about 15 mg/ml or more. In one embodiment, the cannabinoid is present in an amount of about 20 mg/ml or more. In one embodiment, the cannabinoid is present in an amount of about 25 mg/ml or more. In one embodiment, the cannabinoid is present in an amount of about 30 mg/ml or more. In one embodiment, the cannabinoid is present in an amount of about 35 mg/ml or more. In one embodiment, the cannabinoid is present in an amount of about 40 mg/ml or more. In one embodiment, the cannabinoid is present in an amount of about 45 mg/ml or more. In one embodiment, the cannabinoid is present in an amount of about 50 mg/ml or more. In one embodiment, the cannabinoid is present in an amount of about 55 mg/ml or more. In one embodiment, the cannabinoid is present in an amount of about 60 mg/ml or more. In one embodiment, the cannabinoid is present in an amount of about 65 mg/ml or more. In one embodiment, the cannabinoid is present in an amount of about 70 mg/ml or more. In one embodiment, the cannabinoid is present in an amount of about 80 mg/ml or more. In one embodiment, the cannabinoid is present in an amount of about 90 mg/ml or more.

In one embodiment, the one or more stabilizing components are selected from antioxidants, pH modulators, chelators and radical scavengers, and combinations thereof.

In one embodiment, the one or more stabilizing components are selected from the class of compounds selected from enediols, pyrones, monoterpenoids, alpha-keto carboxylates, alpha-hydroxy carboxylic acids, esters, phenolic esters, flavonols o-glycosyls, and combinations thereof.

In one embodiment, the one or more stabilizing components are selected from the group consisting of ascorbic acid, sodium ascorbate, ethyl maltol, thymol, maltol, pyruvic acid, sodium pyruvate, lactic acid, carvacrol, alpha-keto glutaric acid, alpha-keto glutarate salt, triethyl citrate, ethyl vanillate, quercetin, sucrose acetate isobutyrate, retinol, cholecalciferol, vitamin K-hydroquinone, citric acid, tartaric acid, ferulic acid, courmaric acid, propyl gallate, gallic acid, alpha lipoic acid, ascorbyl palmitate, lutein, lycopene, resveratrol, rutin, catechin, carnosol, rosmarinic acid, lipoic acid, α-resorcylic, pyrogallol, malvidin, theaflavin, apigenin, eriodictyol, glycitein, chrysoeriol, kaempferol, luteolin, vitexin, isovitexin, orientin, cannflavin A, cannflavin B, cannflavin C, delphinidin, pelargonidin, epicatechin, myricetin, chrysin, naringenin, α-terpineol, nerol, geranyl acetate, fenchol, propyl gallate, tert-butylhydroquinone, carvone and combinations thereof. In one embodiment, the one or more stabilizing components are ethyl maltol, thymol and pyruvic acid.

In one embodiment, the one or more stabilizing components are one or more antioxidants and one or more chelators.

In one embodiment, the one or more stabilizing components are one or more antioxidants.

The one or more antioxidants may be are selected from the enediol class of compounds. For example, in one embodiment, the one or more antioxidants are selected from the group consisting of ascorbic acid, sodium ascorbate, retinol, cholecalciferol and combinations thereof.

In one embodiment, the one or more antioxidants comprise ascorbic acid and one or more additional antioxidants. In one embodiment, the one or more antioxidants comprise sodium ascorbate and one or more additional antioxidants. In one embodiment, the one or more antioxidants are ascorbic acid and/or sodium ascorbate. In one embodiment, the one or more antioxidants are ascorbic acid and sodium ascorbate. In one embodiment, the antioxidant is ascorbic acid. In one embodiment, the antioxidant is sodium ascorbate.

In one embodiment, the one or more stabilizing components are one or more chelators. In one embodiment, the one or more chelators are selected from the group consisting of ethyl maltol, maltol, and triethyl citrate.

In one embodiment, the one or more stabilizing components are each present in an amount of at least 100 ppm. In one embodiment, the one or more stabilizing components are each present in an amount of at least 200 ppm. In one embodiment, the one or more stabilizing components are each present in an amount of at least 300 ppm. In one embodiment, the one or more stabilizing components are each present in an amount of at least 400 ppm. In one embodiment, the one or more stabilizing components are each present in an amount of at least 500 ppm. In one embodiment, the one or more stabilizing components are each present in an amount of at least 600 ppm. In one embodiment, the one or more stabilizing components are each present in an amount of at least 700 ppm. In one embodiment, the one or more stabilizing components are each present in an amount of at least 800 ppm. In one embodiment, the one or more stabilizing components are each present in an amount of at least 900 ppm. In one embodiment, the one or more stabilizing components are each present in an amount of at least 1000 ppm. In one embodiment, the one or more stabilizing components are each present in an amount of at least 1100 ppm. In one embodiment, the one or more stabilizing components are each present in an amount of at least 1200 ppm. In one embodiment, the one or more stabilizing components are each present in an amount of at least 1300 ppm. In one embodiment, the one or more stabilizing components are each present in an amount of at least 1400 ppm. In one embodiment, the one or more stabilizing components are each present in an amount of at least 1500 ppm. In one embodiment, the one or more stabilizing components are each present in an amount of at least 1600 ppm. In one embodiment, the one or more stabilizing components are each present in an amount of at least 1700 ppm. In one embodiment, the one or more stabilizing components are each present in an amount of at least 1800 ppm. In one embodiment, the one or more stabilizing components are each present in an amount of at least 1900 ppm. In one embodiment, the one or more stabilizing components are each present in an amount of at least 2000 ppm. In one embodiment, the one or more stabilizing components are each present in an amount of at least 2500 ppm. In one embodiment, the one or more stabilizing components are each present in an amount of at least 3000 ppm. In one embodiment, the one or more stabilizing components are each present in an amount of at least 3500 ppm. In one embodiment, the one or more stabilizing components are each present in an amount of at least 4000 ppm. In one embodiment, the one or more stabilizing components are each present in an amount of at least 4500 ppm. In one embodiment, the one or more stabilizing components are each present in an amount of at least 5000 ppm. In one embodiment, the one or more stabilizing components are each present in an amount of at least 6000 ppm. In one embodiment, the one or more stabilizing components are each present in an amount of at least 7000 ppm. In one embodiment, the one or more stabilizing components are each present in an amount of at least 8000 ppm. In one embodiment, the one or more stabilizing components are each present in an amount of at least 9000 ppm. In one embodiment, the one or more stabilizing components are each present in an amount of at least 10000 ppm. In one embodiment, the one or more stabilizing components are each present in an amount of at least 15000 ppm.

In one embodiment, the one or more antioxidants are each present in an amount of at least 500 ppm. In one embodiment, the one or more antioxidants are each present in an amount of at least 600 ppm. In one embodiment, the one or more antioxidants are each present in an amount of at least 700 ppm. In one embodiment, the one or more antioxidants are each present in an amount of at least 800 ppm. In one embodiment, the one or more antioxidants are each present in an amount of at least 900 ppm. In one embodiment, the one or more antioxidants are each present in an amount of at least 1000 ppm. In one embodiment, the one or more antioxidants are each present in an amount of at least 1100 ppm. In one embodiment, the one or more antioxidants are each present in an amount of at least 1200 ppm. In one embodiment, the one or more antioxidants are each present in an amount of at least 1300 ppm. In one embodiment, the one or more antioxidants are each present in an amount of at least 1400 ppm. In one embodiment, the one or more antioxidants are each present in an amount of at least 1500 ppm. In one embodiment, the one or more antioxidants are each present in an amount of at least 1600 ppm. In one embodiment, the one or more antioxidants are each present in an amount of at least 1700 ppm. In one embodiment, the one or more antioxidants are each present in an amount of at least 1800 ppm. In one embodiment, the one or more antioxidants are each present in an amount of at least 1900 ppm. In one embodiment, the one or more antioxidants are each present in an amount of at least 2000 ppm.

In one embodiment, the one or more chelators are each present in an amount of at least 100 ppm. In one embodiment, the one or more chelators are each present in an amount of at least 200 ppm. In one embodiment, the one or more chelators are each present in an amount of at least 300 ppm. In one embodiment, the one or more chelators are each present in an amount of at least 400 ppm. In one embodiment, the one or more chelators are each present in an amount of at least 500 ppm. In one embodiment, the one or more chelators are each present in an amount of at least 6000 ppm. In one embodiment, the one or more chelators are each present in an amount of at least 700 ppm. In one embodiment, the one or more chelators are each present in an amount of at least 800 ppm. In one embodiment, the one or more chelators are each present in an amount of at least 900 ppm. In one embodiment, the one or more chelators are each present in an amount of at least 1000 ppm.

In one embodiment, the one or more antioxidants are each present in an amount of at least 500 ppm and the one or more chelators are each present in an amount of at least 100 ppm. In one embodiment, the one or more antioxidants are each present in an amount of at least 1000 ppm and the one or more chelators are each present in an amount of at least 100 ppm. In one embodiment, the one or more antioxidants are each present in an amount of at least 1500 ppm and the one or more chelators are each present in an amount of at least 100 ppm. In one embodiment, the one or more antioxidants are each present in an amount of at least 2000 ppm and the one or more chelators are each present in an amount of at least 100 ppm. In one embodiment, the one or more antioxidants are each present in an amount of at least 500 ppm and the one or more chelators are each present in an amount of at least 500 ppm. In one embodiment, the one or more antioxidants are each present in an amount of at least 1000 ppm and the one or more chelators are each present in an amount of at least 500 ppm. In one embodiment, the one or more antioxidants are each present in an amount of at least 1500 ppm and the one or more chelators are each present in an amount of at least 500 ppm. In one embodiment, the one or more antioxidants are each present in an amount of at least 2000 ppm and the one or more chelators are each present in an amount of at least 500 ppm. In one embodiment, the one or more antioxidants are each present in an amount of at least 500 ppm and the one or more chelators are each present in an amount of at least 1000 ppm. In one embodiment, the one or more antioxidants are each present in an amount of at least 1000 ppm and the one or more chelators are each present in an amount of at least 1000 ppm. In one embodiment, the one or more antioxidants are each present in an amount of at least 1500 ppm and the one or more chelators are each present in an amount of at least 1000 ppm. In one embodiment, the one or more antioxidants are each present in an amount of at least 2000 ppm and the one or more chelators are each present in an amount of at least 1000 ppm.

In one embodiment, the one or more antioxidants comprise ascorbic acid and one or more additional antioxidants, wherein ascorbic acid is present in an amount of at least 400 ppm. In one embodiment, the one or more antioxidants comprise ascorbic acid and one or more additional antioxidants, wherein ascorbic acid is present in an amount of at least 500 ppm. In one embodiment, the one or more antioxidants comprise ascorbic acid and one or more additional antioxidants, wherein ascorbic acid is present in an amount of at least 750 ppm. In one embodiment, the one or more antioxidants comprise ascorbic acid and one or more additional antioxidants, wherein ascorbic acid is present in an amount of at least 1000 ppm. In one embodiment, the one or more antioxidants comprise ascorbic acid and one or more additional antioxidants, wherein ascorbic acid is present in an amount of at least 1250 ppm. In one embodiment, the one or more antioxidants comprise ascorbic acid and one or more additional antioxidants, wherein ascorbic acid is present in an amount of at least 1500 ppm. In one embodiment, the one or more antioxidants comprise ascorbic acid and one or more additional antioxidants, wherein ascorbic acid is present in an amount of at least 1750 ppm. In one embodiment, the one or more antioxidants comprise ascorbic acid and one or more additional antioxidants, wherein ascorbic acid is present in an amount of at least 2000 ppm. In one embodiment, the one or more antioxidants comprise ascorbic acid and one or more additional antioxidants, wherein ascorbic acid is present in an amount of at least 2500 ppm. In one embodiment, the one or more antioxidants comprise ascorbic acid and one or more additional antioxidants, wherein ascorbic acid is present in an amount of at least 3000 ppm. In one embodiment, the one or more antioxidants comprise ascorbic acid and one or more additional antioxidants, wherein ascorbic acid is present in an amount of at least 3500 ppm. In one embodiment, the one or more antioxidants comprise ascorbic acid and one or more additional antioxidants, wherein ascorbic acid is present in an amount of at least 4000 ppm. In one embodiment, the one or more antioxidants comprise ascorbic acid and one or more additional antioxidants, wherein ascorbic acid is present in an amount of at least 4500 ppm. In one embodiment, the one or more antioxidants comprise ascorbic acid and one or more additional antioxidants, wherein ascorbic acid is present in an amount of at least 5000 ppm. In one embodiment, the one or more antioxidants comprise ascorbic acid and one or more additional antioxidants, wherein ascorbic acid is present in an amount of at least 6000 ppm. In one embodiment, the one or more antioxidants comprise ascorbic acid and one or more additional antioxidants, wherein ascorbic acid is present in an amount of at least 7000 ppm.

In one embodiment, the antioxidants are ascorbic acid and sodium ascorbate, wherein ascorbic acid is present in an amount of at least 500 ppm and sodium ascorbate is present in an amount of at least 500 ppm. In one embodiment, the antioxidants are ascorbic acid and sodium ascorbate, wherein ascorbic acid is present in an amount of at least 750 ppm and sodium ascorbate is present in an amount of at least 500 ppm. In one embodiment, the antioxidants are ascorbic acid and sodium ascorbate, wherein ascorbic acid is present in an amount of at least 1000 ppm and sodium ascorbate is present in an amount of at least 500 ppm. In one embodiment, the antioxidants are ascorbic acid and sodium ascorbate, wherein ascorbic acid is present in an amount of at least 1250 ppm and sodium ascorbate is present in an amount of at least 500 ppm. In one embodiment, the antioxidants are ascorbic acid and sodium ascorbate, wherein ascorbic acid is present in an amount of at least 1500 ppm and sodium ascorbate is present in an amount of at least 500 ppm. In one embodiment, the antioxidants are ascorbic acid and sodium ascorbate, wherein ascorbic acid is present in an amount of at least 1750 ppm and sodium ascorbate is present in an amount of at least 500 ppm. In one embodiment, the antioxidants are ascorbic acid and sodium ascorbate, wherein ascorbic acid is present in an amount of at least 2000 ppm and sodium ascorbate is present in an amount of at least 500 ppm. In one embodiment, the antioxidants are ascorbic acid and sodium ascorbate, wherein ascorbic acid is present in an amount of at least 500 ppm and sodium ascorbate is present in an amount of at least 750 ppm. In one embodiment, the antioxidants are ascorbic acid and sodium ascorbate, wherein ascorbic acid is present in an amount of at least 750 ppm and sodium ascorbate is present in an amount of at least 750 ppm. In one embodiment, the antioxidants are ascorbic acid and sodium ascorbate, wherein ascorbic acid is present in an amount of at least 1000 ppm and sodium ascorbate is present in an amount of at least 750 ppm. In one embodiment, the antioxidants are ascorbic acid and sodium ascorbate, wherein ascorbic acid is present in an amount of at least 1250 ppm and sodium ascorbate is present in an amount of at least 750 ppm. In one embodiment, the antioxidants are ascorbic acid and sodium ascorbate, wherein ascorbic acid is present in an amount of at least 1500 ppm and sodium ascorbate is present in an amount of at least 750 ppm. In one embodiment, the antioxidants are ascorbic acid and sodium ascorbate, wherein ascorbic acid is present in an amount of at least 1750 ppm and sodium ascorbate is present in an amount of at least 750 ppm. In one embodiment, the antioxidants are ascorbic acid and sodium ascorbate, wherein ascorbic acid is present in an amount of at least 2000 ppm and sodium ascorbate is present in an amount of at least 750 ppm. In one embodiment, the antioxidants are ascorbic acid and sodium ascorbate, wherein ascorbic acid is present in an amount of at least 1000 ppm and sodium ascorbate is present in an amount of at least 1000 ppm. In one embodiment, the antioxidants are ascorbic acid and sodium ascorbate, wherein ascorbic acid is present in an amount of at least 1250 ppm and sodium ascorbate is present in an amount of at least 1000 ppm. In one embodiment, the antioxidants are ascorbic acid and sodium ascorbate, wherein ascorbic acid is present in an amount of at least 1500 ppm and sodium ascorbate is present in an amount of at least 1000 ppm. In one embodiment, the antioxidants are ascorbic acid and sodium ascorbate, wherein ascorbic acid is present in an amount of at least 1750 ppm and sodium ascorbate is present in an amount of at least 1000 ppm. In one embodiment, the antioxidants are ascorbic acid and sodium ascorbate, wherein ascorbic acid is present in an amount of at least 2000 ppm and sodium ascorbate is present in an amount of at least 1000 ppm. In one embodiment, the antioxidants are ascorbic acid and sodium ascorbate, wherein ascorbic acid is present in an amount of at least 1000 ppm and sodium ascorbate is present in an amount of at least 2000 ppm. In one embodiment, the antioxidants are ascorbic acid and sodium ascorbate, wherein ascorbic acid is present in an amount of at least 2000 ppm and sodium ascorbate is present in an amount of at least 2000 ppm. In one embodiment, the antioxidants are ascorbic acid and sodium ascorbate, wherein ascorbic acid is present in an amount of at least 3000 ppm and sodium ascorbate is present in an amount of at least 2000 ppm. In one embodiment, the antioxidants are ascorbic acid and sodium ascorbate, wherein ascorbic acid is present in an amount of at least 4000 ppm and sodium ascorbate is present in an amount of at least 2000 ppm. In one embodiment, the antioxidants are ascorbic acid and sodium ascorbate, wherein ascorbic acid is present in an amount of at least 1000 ppm and sodium ascorbate is present in an amount of at least 3000 ppm. In one embodiment, the antioxidants are ascorbic acid and sodium ascorbate, wherein ascorbic acid is present in an amount of at least 2000 ppm and sodium ascorbate is present in an amount of at least 3000 ppm. In one embodiment, the antioxidants are ascorbic acid and sodium ascorbate, wherein ascorbic acid is present in an amount of at least 3000 ppm and sodium ascorbate is present in an amount of at least 3000 ppm. In one embodiment, the antioxidants are ascorbic acid and sodium ascorbate, wherein ascorbic acid is present in an amount of at least 4000 ppm and sodium ascorbate is present in an amount of at least 3000 ppm. In one embodiment, the antioxidants are ascorbic acid and sodium ascorbate, wherein ascorbic acid is present in an amount of at least 1000 ppm and sodium ascorbate is present in an amount of at least 4000 ppm. In one embodiment, the antioxidants are ascorbic acid and sodium ascorbate, wherein ascorbic acid is present in an amount of at least 2000 ppm and sodium ascorbate is present in an amount of at least 4000 ppm. In one embodiment, the antioxidants are ascorbic acid and sodium ascorbate, wherein ascorbic acid is present in an amount of at least 3000 ppm and sodium ascorbate is present in an amount of at least 4000 ppm. In one embodiment, the antioxidants are ascorbic acid and sodium ascorbate, wherein ascorbic acid is present in an amount of at least 4000 ppm and sodium ascorbate is present in an amount of at least 4000 ppm.

In one embodiment, the antioxidants are ascorbic acid and sodium ascorbate, wherein ascorbic acid is present in an amount of from 500 to 4000 ppm and sodium ascorbate is present in an amount of from 500 to 4000 ppm. In one embodiment, the antioxidants are ascorbic acid and sodium ascorbate, wherein ascorbic acid is present in an amount of from 500 to 4000 ppm and sodium ascorbate is present in an amount of from 1000 to 3000 ppm.

In one embodiment, the antioxidants are ascorbic acid and sodium ascorbate, wherein ascorbic acid is present in an amount of from 500 to 4000 ppm and sodium ascorbate is present in an amount of from 1000 to 2000 ppm. In one embodiment, the antioxidants are ascorbic acid and sodium ascorbate, wherein ascorbic acid is present in an amount of from 1000 to 3000 ppm and sodium ascorbate is present in an amount of from 500 to 4000 ppm. In one embodiment, the antioxidants are ascorbic acid and sodium ascorbate, wherein ascorbic acid is present in an amount of from 1000 to 2000 ppm and sodium ascorbate is present in an amount of from 500 to 4000 ppm.

In one embodiment, the antioxidants are ascorbic acid and sodium ascorbate, wherein ascorbic acid is present in an amount of from 1000 to 4000 ppm and sodium ascorbate is present in an amount of from 1000 to 4000 ppm. In one embodiment, the antioxidants are ascorbic acid and sodium ascorbate, wherein ascorbic acid is present in an amount of from 1000 to 3000 ppm and sodium ascorbate is present in an amount of from 1000 to 3000 ppm. In one embodiment, the antioxidants are ascorbic acid and sodium ascorbate, wherein ascorbic acid is present in an amount of from 1000 to 2000 ppm and sodium ascorbate is present in an amount of from 1000 to 2000 ppm. In one embodiment, the antioxidants are ascorbic acid and sodium ascorbate, wherein ascorbic acid is present in an amount of from 1000 to 2000 ppm and sodium ascorbate is present in an amount of from 250 to 1000 ppm. In one embodiment, the antioxidants are ascorbic acid and sodium ascorbate, wherein ascorbic acid is present in an amount of from 1000 to 1750 ppm and sodium ascorbate is present in an amount of from 250 to 1000 ppm. In one embodiment, the antioxidants are ascorbic acid and sodium ascorbate, wherein ascorbic acid is present in an amount of from 1000 to 1500 ppm and sodium ascorbate is present in an amount of from 250 to 1000 ppm. In one embodiment, the antioxidants are ascorbic acid and sodium ascorbate, wherein ascorbic acid is present in an amount of from 1000 to 1250 ppm and sodium ascorbate is present in an amount of from 250 to 1000 ppm. In one embodiment, the antioxidants are ascorbic acid and sodium ascorbate, wherein ascorbic acid is present in an amount of from 1000 to 2000 ppm and sodium ascorbate is present in an amount of from 500 to 1000 ppm. In one embodiment, the antioxidants are ascorbic acid and sodium ascorbate, wherein ascorbic acid is present in an amount of from 1000 to 1750 ppm and sodium ascorbate is present in an amount of from 500 to 1000 ppm. In one embodiment, the antioxidants are ascorbic acid and sodium ascorbate, wherein ascorbic acid is present in an amount of from 1000 to 1500 ppm and sodium ascorbate is present in an amount of from 500 to 1000 ppm. In one embodiment, the antioxidants are ascorbic acid and sodium ascorbate, wherein ascorbic acid is present in an amount of from 1000 to 1250 ppm and sodium ascorbate is present in an amount of from 500 to 1000 ppm. In one embodiment, the antioxidants are ascorbic acid and sodium ascorbate, wherein ascorbic acid is present in an amount of from 1000 to 2000 ppm and sodium ascorbate is present in an amount of from 750 to 1000 ppm. In one embodiment, the antioxidants are ascorbic acid and sodium ascorbate, wherein ascorbic acid is present in an amount of from 1000 to 1750 ppm and sodium ascorbate is present in an amount of from 750 to 1000 ppm. In one embodiment, the antioxidants are ascorbic acid and sodium ascorbate, wherein ascorbic acid is present in an amount of from 1000 to 1500 ppm and sodium ascorbate is present in an amount of from 750 to 1000 ppm. In one embodiment, the antioxidants are ascorbic acid and sodium ascorbate, wherein ascorbic acid is present in an amount of from 1000 to 1250 ppm and sodium ascorbate is present in an amount of from 750 to 1000 ppm. In one embodiment, the antioxidants are ascorbic acid and sodium ascorbate, wherein ascorbic acid is present in an amount of from 750 to 2000 ppm and sodium ascorbate is present in an amount of from 750 to 1250 ppm. In one embodiment, the antioxidants are ascorbic acid and sodium ascorbate, wherein ascorbic acid is present in an amount of from 750 to 1750 ppm and sodium ascorbate is present in an amount of from 750 to 1250 ppm. In one embodiment, the antioxidants are ascorbic acid and sodium ascorbate, wherein ascorbic acid is present in an amount of from 750 to 1500 ppm and sodium ascorbate is present in an amount of from 750 to 1250 ppm. In one embodiment, the antioxidants are ascorbic acid and sodium ascorbate, wherein ascorbic acid is present in an amount of from 750 to 1250 ppm and sodium ascorbate is present in an amount of from 750 to 1250 ppm. In one embodiment, the antioxidants are ascorbic acid and sodium ascorbate, wherein ascorbic acid is present in an amount of from 750 to 1250 ppm and sodium ascorbate is present in an amount of from 750 to 2000 ppm. In one embodiment, the antioxidants are ascorbic acid and sodium ascorbate, wherein ascorbic acid is present in an amount of from 750 to 1250 ppm and sodium ascorbate is present in an amount of from 750 to 1750 ppm. In one embodiment, the antioxidants are ascorbic acid and sodium ascorbate, wherein ascorbic acid is present in an amount of from 750 to 1250 ppm and sodium ascorbate is present in an amount of from 750 to 1500 ppm. In one embodiment, the antioxidants are ascorbic acid and sodium ascorbate, wherein ascorbic acid is present in an amount of about 1750 ppm and sodium ascorbate is present in an amount of about 250 ppm. In one embodiment, the antioxidants are ascorbic acid and sodium ascorbate, wherein ascorbic acid is present in an amount of about 1500 ppm and sodium ascorbate is present in an amount of about 500 ppm. In one embodiment, the antioxidants are ascorbic acid and sodium ascorbate, wherein ascorbic acid is present in an amount of about 1250 ppm and sodium ascorbate is present in an amount of about 750 ppm. In one embodiment, the antioxidants are ascorbic acid and sodium ascorbate, wherein ascorbic acid is present in an amount of about 1000 ppm and sodium ascorbate is present in an amount of about 1000 ppm.

In one embodiment, the one or more stabilizing components are present in an amount of from 0.05% w/w to 10% w/w based on the total weight of the formulation. In one embodiment, the one or more stabilizing components are present in an amount of from 0.05% w/w to 9% w/w based on the total weight of the formulation. In one embodiment, the one or more stabilizing components are present in an amount of from 0.05% w/w to 8% w/w based on the total weight of the formulation. In one embodiment, the one or more stabilizing components are present in an amount of from 0.05% w/w to 7% w/w based on the total weight of the formulation. In one embodiment, the one or more stabilizing components are present in an amount of from 0.05% w/w to 6% w/w based on the total weight of the formulation. In one embodiment, the one or more stabilizing components are present in an amount of from 0.05% w/w to 5% w/w based on the total weight of the formulation. In one embodiment, the one or more stabilizing components are present in an amount of from 0.05% w/w to 4% w/w based on the total weight of the formulation. In one embodiment, the one or more stabilizing components are present in an amount of from 0.05% w/w to 3% w/w based on the total weight of the formulation. In one embodiment, the one or more stabilizing components are present in an amount of from 0.05% w/w to 2% w/w based on the total weight of the formulation. In one embodiment, the one or more stabilizing components are present in an amount of from 0.05% w/w to 1% w/w based on the total weight of the formulation. In one embodiment, the one or more stabilizing components are present in an amount of from 0.05% w/w to 0.5% w/w based on the total weight of the formulation. In one embodiment, the one or more stabilizing components are present in an amount of from 0.05% w/w to 0.4% w/w based on the total weight of the formulation. In one embodiment, the one or more stabilizing components are present in an amount of from 0.05% w/w to 0.3% w/w based on the total weight of the formulation. In one embodiment, the one or more stabilizing components are present in an amount of from 0.05% w/w to 0.2% w/w based on the total weight of the formulation. In one embodiment, the one or more stabilizing components are present in an amount of from 0.05% w/w to 0.15% w/w based on the total weight of the formulation. In one embodiment, the one or more stabilizing components are present in an amount of from 0.1% w/w to 0.15% w/w based on the total weight of the formulation. In one embodiment, the one or more stabilizing components are present in an amount of about 0.12% w/w based on the total weight of the formulation.

In one embodiment, the cannabinoids comprise CBD and wherein CBD and the one or more antioxidants are present in a molar ratio of from 1:1 (cannabinoid to antioxidant) to 55:1 (cannabinoid to antioxidant).

In one embodiment, the cannabinoids comprise CBD and wherein CBD and the one or more antioxidants are present in a molar ratio of at least 50:1 (CBD to antioxidant). In one embodiment, the cannabinoids comprise CBD and wherein CBD and the one or more antioxidants are present in a molar ratio of at least 40:1. In one embodiment, the cannabinoids comprise CBD and wherein CBD and the one or more antioxidants are present in a molar ratio of at least 35:1. In one embodiment, the cannabinoids comprise CBD and wherein CBD and the one or more antioxidants are present in a molar ratio of at least 30:1. In one embodiment, the cannabinoids comprise CBD and wherein CBD and the one or more antioxidants are present in a molar ratio of at least 25:1. In one embodiment, the cannabinoids comprise CBD and wherein CBD and the one or more antioxidants are present in a molar ratio of at least 20:1. In one embodiment, the cannabinoids comprise CBD and wherein CBD and the one or more antioxidants are present in a molar ratio of at least 15:1. In one embodiment, the cannabinoids comprise CBD and wherein CBD and the one or more antioxidants are present in a molar ratio of at least 10:1. In one embodiment, the cannabinoids comprise CBD and wherein CBD and the one or more antioxidants are present in a molar ratio of at least 8:1. In one embodiment, the cannabinoids comprise CBD and wherein CBD and the one or more antioxidants are present in a molar ratio of at least 6:1. In one embodiment, the cannabinoids comprise CBD and wherein CBD and the one or more antioxidants are present in a molar ratio of at least 5:1. In one embodiment, the cannabinoids comprise CBD and wherein CBD and the one or more antioxidants are present in a molar ratio of at least 4:1. In one embodiment, the cannabinoids comprise CBD and wherein CBD and the one or more antioxidants are present in a molar ratio of at least 3:1.

In one embodiment, the cannabinoids comprise CBD and the antioxidants comprise ascorbic acid, wherein CBD and ascorbic acid are present in a molar ratio of at least 50:1 (CBD to ascorbic acid). In one embodiment, the cannabinoids comprise CBD and the antioxidants comprise ascorbic acid, wherein CBD and ascorbic acid are present in a molar ratio of at least 40:1. In one embodiment, the cannabinoids comprise CBD and the antioxidants comprise ascorbic acid, wherein CBD and ascorbic acid are present in a molar ratio of at least 30:1. In one embodiment, the cannabinoids comprise CBD and the antioxidants comprise ascorbic acid, wherein CBD and ascorbic acid are present in a molar ratio of at least 20:1. In one embodiment, the cannabinoids comprise CBD and the antioxidants comprise ascorbic acid, wherein CBD and ascorbic acid are present in a molar ratio of at least 15:1. In one embodiment, the cannabinoids comprise CBD and the antioxidants comprise ascorbic acid, wherein CBD and ascorbic acid are present in a molar ratio of at least 10:1. In one embodiment, the cannabinoids comprise CBD and the antioxidants comprise ascorbic acid, wherein CBD and ascorbic acid are present in a molar ratio of at least 8:1. In one embodiment, the cannabinoids comprise CBD and the antioxidants comprise ascorbic acid, wherein CBD and ascorbic acid are present in a molar ratio of at least 6:1. In one embodiment, the cannabinoids comprise CBD and the antioxidants comprise ascorbic acid, wherein CBD and ascorbic acid are present in a molar ratio of at least 4:1. In one embodiment, the cannabinoids comprise CBD and the antioxidants comprise ascorbic acid, wherein CBD and ascorbic acid are present in a molar ratio of at least 3:1. In one embodiment, the cannabinoids comprise CBD and the antioxidants comprise ascorbic acid, wherein CBD and ascorbic acid are present in a molar ratio of at least 2:1. In one embodiment, the cannabinoids comprise CBD and the antioxidants comprise ascorbic acid, wherein CBD and ascorbic acid are present in a molar ratio of at least 2:1.

In one embodiment, the cannabinoids comprise CBD and the antioxidants comprise sodium ascorbate, wherein CBD and sodium ascorbate are present in a molar ratio of at least 50:1 (CBD to sodium ascorbate). In one embodiment, the cannabinoids comprise CBD and the antioxidants comprise sodium ascorbate, wherein CBD and sodium ascorbate are present in a molar ratio of at least 40:1. In one embodiment, the cannabinoids comprise CBD and the antioxidants comprise sodium ascorbate, wherein CBD and sodium ascorbate are present in a molar ratio of at least 30:1. In one embodiment, the cannabinoids comprise CBD and the antioxidants comprise sodium ascorbate, wherein CBD and sodium ascorbate are present in a molar ratio of at least 20:1. In one embodiment, the cannabinoids comprise CBD and the antioxidants comprise sodium ascorbate, wherein CBD and sodium ascorbate are present in a molar ratio of at least 15:1. In one embodiment, the cannabinoids comprise CBD and the antioxidants comprise sodium ascorbate, wherein CBD and sodium ascorbate are present in a molar ratio of at least 10:1. In one embodiment, the cannabinoids comprise CBD and the antioxidants comprise sodium ascorbate, wherein CBD and sodium ascorbate are present in a molar ratio of at least 8:1. In one embodiment, the cannabinoids comprise CBD and the antioxidants comprise sodium ascorbate, wherein CBD and sodium ascorbate are present in a molar ratio of at least 6:1. In one embodiment, the cannabinoids comprise CBD and the antioxidants comprise sodium ascorbate, wherein CBD and sodium ascorbate are present in a molar ratio of at least 4:1. In one embodiment, the cannabinoids comprise CBD and the antioxidants comprise sodium ascorbate, wherein CBD and sodium ascorbate are present in a molar ratio of at least 3:1. In one embodiment, the cannabinoids comprise CBD and the antioxidants comprise sodium ascorbate, wherein CBD and sodium ascorbate are present in a molar ratio of at least 2:1. In one embodiment, the cannabinoids comprise CBD and the antioxidants comprise sodium ascorbate, wherein CBD and sodium ascorbate are present in a molar ratio of at least 1:1.

The carrier constituent comprises one or more constituents capable of forming an aerosol, particularly when evaporated and allowed to condense. In some embodiments, the carrier constituent may comprise one or more of glycerol, propylene glycol, triethylene glycol, tetraethylene glycol, 1,3-butylene glycol, erythritol, meso-Erythritol, ethyl laurate, a diethyl suberatetriethylene glycol diacetate, triacetin, a diacetin mixture, benzyl benzoate, benzyl phenyl acetate, tributyrin, lauryl acetate, lauric acid, myristic acid, and propylene carbonate.

In one embodiment, the total amount of carrier constituents is 55% w/w or more based on the total weight of the formulation. In one embodiment, the total amount of carrier constituents is 60% w/w or more based on the total weight of the formulation. In one embodiment, the total amount of carrier constituents is 65% w/w or more based on the total weight of the formulation. In one embodiment, the total amount of carrier constituents is 70% w/w or more based on the total weight of the formulation. In one embodiment, the total amount of carrier constituents is 75% w/w or more based on the total weight of the formulation. In one embodiment, the total amount of carrier constituents is 80% w/w or more based on the total weight of the formulation. In one embodiment, the total amount of carrier constituents is 85% w/w or more based on the total weight of the formulation. In one embodiment, the total amount of carrier constituents is 90% w/w or more based on the total weight of the formulation. In one embodiment, the total amount of carrier constituents is 95% w/w or more based on the total weight of the formulation.

In one embodiment, the carrier constituent comprises propylene glycol.

In one embodiment, propylene glycol is present in an amount of from 10% w/w to 95% w/w based on the total weight of the formulation. In one embodiment, propylene glycol is present in an amount of from 20% w/w to 95% w/w based on the total weight of the formulation. In one embodiment, propylene glycol is present in an amount of from 30% w/w to 95% w/w based on the total weight of the formulation. In one embodiment, propylene glycol is present in an amount of from 40% w/w to 95% w/w based on the total weight of the formulation.

In one embodiment, propylene glycol is present in an amount of from 50% w/w to 90% w/w based on the total weight of the formulation. In one embodiment, propylene glycol is present in an amount of from 50% w/w to 85% w/w based on the total weight of the formulation. In one embodiment, propylene glycol is present in an amount of from 50% w/w to 80% w/w based on the total weight of the formulation. In one embodiment, propylene glycol is present in an amount of from 50% w/w to 75% w/w based on the total weight of the formulation. In one embodiment, propylene glycol is present in an amount of from 50% w/w to 60% w/w based on the total weight of the formulation. In one embodiment, propylene glycol is present in an amount of from 50% w/w to 65% w/w based on the total weight of the formulation. In one embodiment, propylene glycol is present in an amount of from 50% w/w to 60% w/w based on the total weight of the formulation.

In one embodiment, propylene glycol is present in an amount of from 55% w/w to 90% w/w based on the total weight of the formulation. In one embodiment, propylene glycol is present in an amount of from 60% w/w to 90% w/w based on the total weight of the formulation. In one embodiment, propylene glycol is present in an amount of from 65% w/w to 90% w/w based on the total weight of the formulation. In one embodiment, propylene glycol is present in an amount of from 70% w/w to 90% w/w based on the total weight of the formulation. In one embodiment, propylene glycol is present in an amount of from 75% w/w to 90% w/w based on the total weight of the formulation. In one embodiment, propylene glycol is present in an amount of from 80% w/w to 90% w/w based on the total weight of the formulation. In one embodiment, propylene glycol is present in an amount of from 85% w/w to 90% w/w based on the total weight of the formulation.

In one embodiment, propylene glycol is present in an amount of at least 10% w/w based on the total weight of the formulation. In one embodiment, propylene glycol is present in an amount of at least 20% w/w based on the total weight of the formulation. In one embodiment, propylene glycol is present in an amount of at least 30% w/w based on the total weight of the formulation. In one embodiment, propylene glycol is present in an amount of at least 40% w/w based on the total weight of the formulation. In one embodiment, propylene glycol is present in an amount of at least 50% w/w based on the total weight of the formulation. In one embodiment, propylene glycol is present in an amount of at least 55% w/w based on the total weight of the formulation. In one embodiment, propylene glycol is present in an amount of at least 60% w/w based on the total weight of the formulation. In one embodiment, propylene glycol is present in an amount of at least 65% w/w based on the total weight of the formulation. In one embodiment, propylene glycol is present in an amount of at least 70% w/w based on the total weight of the formulation. In one embodiment, propylene glycol is present in an amount of at least 75% w/w based on the total weight of the formulation. In one embodiment, propylene glycol is present in an amount of at least 80% w/w based on the total weight of the formulation. In one embodiment, propylene glycol is present in an amount of at least 85% w/w based on the total weight of the formulation. In one embodiment, propylene glycol is present in an amount of at least 90% w/w based on the total weight of the formulation.

In one embodiment, propylene glycol is present in an amount of about 70% w/w.

In some embodiments, the w/w % amount of propylene glycol in the formulation, based on the total weight of the formulation, is equal to or above a threshold C_{%}, the threshold being defined according to

C_{%}=11.416×(A)^{0.377}

wherein A is the amount of the at least one cannabinoid present in the formulation in mg/ml. It has been found that formulations comprising at least one cannabinoid, such as cannabidiol, and propylene glycol conforming to the above threshold, are particularly stable.

In one embodiment, the carrier constituent comprises glycerol.

In one embodiment, glycerol is present in an amount of from 10% w/w to 95% w/w based on the total weight of the formulation. In one embodiment, glycerol is present in an amount of from 20% w/w to 95% w/w based on the total weight of the formulation. In one embodiment, glycerol is present in an amount of from 30% w/w to 95% w/w based on the total weight of the formulation. In one embodiment, glycerol is present in an amount of from 40% w/w to 95% w/w based on the total weight of the formulation. In one embodiment, glycerol is present in an amount of from 50% w/w to 95% w/w based on the total weight of the formulation.

In one embodiment, glycerol is present in an amount of from 50% w/w to 90% w/w based on the total weight of the formulation. In one embodiment, glycerol is present in an amount of from 50% w/w to 85% w/w based on the total weight of the formulation. In one embodiment, glycerol is present in an amount of from 50% w/w to 80% w/w based on the total weight of the formulation. In one embodiment, glycerol is present in an amount of from 50% w/w to 75% w/w based on the total weight of the formulation. In one embodiment, glycerol is present in an amount of from 50% w/w to 60% w/w based on the total weight of the formulation. In one embodiment, glycerol is present in an amount of from 50% w/w to 65% w/w based on the total weight of the formulation. In one embodiment, glycerol is present in an amount of from 50% w/w to 60% w/w based on the total weight of the formulation.

In one embodiment, glycerol is present in an amount of from 55% w/w to 90% w/w based on the total weight of the formulation. In one embodiment, glycerol is present in an amount of from 60% w/w to 90% w/w based on the total weight of the formulation. In one embodiment, glycerol is present in an amount of from 65% w/w to 90% w/w based on the total weight of the formulation. In one embodiment, glycerol is present in an amount of from 70% w/w to 90% w/w based on the total weight of the formulation. In one embodiment, glycerol is present in an amount of from 75% w/w to 90% w/w based on the total weight of the formulation. In one embodiment, glycerol is present in an amount of from 80% w/w to 90% w/w based on the total weight of the formulation. In one embodiment, glycerol is present in an amount of from 85% w/w to 90% w/w based on the total weight of the formulation.

In one embodiment, glycerol is present in an amount of at least 10% w/w based on the total weight of the formulation. In one embodiment, glycerol is present in an amount of at least 20% w/w based on the total weight of the formulation. In one embodiment, glycerol is present in an amount of at least 30% w/w based on the total weight of the formulation. In one embodiment, glycerol is present in an amount of at least 40% w/w based on the total weight of the formulation. In one embodiment, glycerol is present in an amount of at least 50% w/w based on the total weight of the formulation. In one embodiment, glycerol is present in an amount of at least 50% w/w based on the total weight of the formulation. In one embodiment, glycerol is present in an amount of at least 55% w/w based on the total weight of the formulation. In one embodiment, glycerol is present in an amount of at least 60% w/w based on the total weight of the formulation. In one embodiment, glycerol is present in an amount of at least 65% w/w based on the total weight of the formulation. In one embodiment, glycerol is present in an amount of at least 70% w/w based on the total weight of the formulation. In one embodiment, glycerol is present in an amount of at least 75% w/w based on the total weight of the formulation. In one embodiment, glycerol is present in an amount of at least 80% w/w based on the total weight of the formulation. In one embodiment, glycerol is present in an amount of at least 85% w/w based on the total weight of the formulation. In one embodiment, glycerol is present in an amount of at least 90% w/w based on the total weight of the formulation.

In one embodiment, both glycerol and propylene glycol are present as carrier constituents.

In one embodiment, glycerol and propylene glycol are present in the formulation in the following amounts:
60 to 90% w/w propylene glycol; and
40 to 10% w/w glycerol, based on the total weight of glycerol and propylene glycol present in the formulation.

In one embodiment, glycerol and propylene glycol are present in the formulation in the following amounts:
70 to 80% w/w propylene glycol; and
30 to 20% w/w glycerol.
   based on the total weight of glycerol and propylene glycol present in the formulation.

In one embodiment, the formulation comprises about 70% w/w propylene glycol and about 30% glycerol.

In one embodiment, the formulation is a liquid at about 25° C.

The formulation may comprise one or more further constituents. In particular, one or more further constituents may be selected from one or more physiologically and/or olfactory active constituents, and/or one or more functional constituents.

In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavor” and/or “flavorant” which, where local regulations permit, may be used to create a desired taste, aroma or sensation in a product for adult consumers. In some instances such constituents may be referred to as flavors, flavorants, cooling agents, heating agents, or sweetening agents, and may include one or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

The flavor may be added to the formulation as part of a so-called “flavor block”, where one or more flavors are blended together and then added to the formulation.

In some embodiments, the formulation may comprise one or more terpenes. For example, the olfactory active constituent may comprise one or more terpenes. In some embodiments, the terpene is a terpene derivable from a phytocannabinoid producing plant, such as a plant from the strain of the cannabis sativa species, such as hemp.

Suitable terpenes in this regard include so-called “C10” terpenes, which are those terpenes comprising 10 carbon atoms, and so-called “C15” terpenes, which are those terpenes comprising 15 carbon atoms.

In some embodiments, the formulation comprises more than one terpene. For example, the formulation may comprise one, two, three, four, five, six, seven, eight, nine, ten or more terpenes as defined herein.

In some embodiments, the terpene is selected based on its solubility in a propylene glycol/glycerol system.

In this regard, the terpene may be selected on the basis of being soluble when present in a propylene glycol/glycerol system, where the w/w % amount of propylene glycol C_{%} present in the formulation, based on the total weight of the formulation, is determined on the basis of the following relationship:

C_{%}=11.416×(T)^{0.377}

wherein T is the amount of the at least one terpene present in the formulation in mg/ml.

By ensuring the selected terpene meets the above threshold when present in a propylene glycol/glycerol system, the stability of the system will not be substantially compromised by including a terpene. In other words, the terpene(s) may be selected such that their solubility in propylene glycol is substantially matched to that of cannabidiol.

In some embodiments, the terpene is selected from pinene (alpha and beta), geraniol, linalool, limonene, eucalyptol, menthone, iso-menthone, piperitone, myrcene, beta-bourbonene, germacrene and mixtures thereof.

In some embodiments, the formulation comprises a combination of terpenes. In some embodiments, the combination of terpenes may comprise a combination of at least geraniol and linalool. In some embodiments, the combination of terpenes may comprise a combination of at least eucalyptol and menthone. In some embodiments, the combination of terpenes may comprise a combination of at least eucalyptol, carvone, piperitone and menthone. In some embodiments, the combination of terpenes may comprise a combination of at least eucalyptol, carvone, beta-bourbonene, germacrene, piperitone, iso-menthone and menthone.

In one embodiment, the terpene(s) are present in a flavour block. This means that the terpenes are blended with one or more other flavours (optionally with an appropriate solvent, for example propylene glycol) and then the flavour block is added during the manufacture of the formulation. In some embodiments, the total amount of the flavour block present in the formulation is up to about 10 w/w %. In some embodiments, the total amount of the flavour block present in the formulation is up to about 9 w/w %. In some embodiments, the total amount of the flavour block present in the formulation is up to about 8 w/w %. In some embodiments, the total amount of the flavour block present in the formulation is up to about 7 w/w %. In some embodiments, the total amount of the flavour block present in the formulation is up to about 6 w/w %. In some embodiments, the total amount of the flavour block present in the formulation is up to about 5 w/w %.

In one embodiment, the total amount of terpene present in the formulation is up to about 10 mg/ml. In one embodiment, the total amount of terpene present in the formulation is up to about 9 mg/ml. In one embodiment, the total amount of terpene present in the formulation is up to about 8 mg/ml. In one embodiment, the total amount of terpene present in the formulation is up to about 7 mg/ml. In one embodiment, the total amount of terpene present in the formulation is up to about 6 mg/ml. In one embodiment, the total amount of terpene present in the formulation is up to about 5 mg/ml. In one embodiment, the total amount of terpene present in the formulation is up to about 4 mg/ml. In one embodiment, the total amount of terpene present in the formulation is up to about 3 mg/ml. In one embodiment, the total amount of terpene present in the formulation is up to about 2 mg/ml. In one embodiment, the total amount of terpene present in the formulation is up to about 1 mg/ml.

In one embodiment, the total amount of terpene present in the formulation is from about 0.1 mg/ml up to about 10 mg/ml. In one embodiment, the total amount of terpene present in the formulation is from about 0.2 mg/ml up to about 10 mg/ml. In one embodiment, the total amount of terpene present in the formulation is from about 0.3 mg/ml up to about 10 mg/ml. In one embodiment, the total amount of terpene present in the formulation is from about 0.4 mg/ml up to about 10 mg/ml. In one embodiment, the total amount of terpene present in the formulation is from about 0.5 mg/ml up to about 10 mg/ml. In one embodiment, the total amount of terpene present in the formulation is from about 1.0 mg/ml up to about 10 mg/ml. In one embodiment, the total amount of terpene present in the formulation is from about 2.0 mg/ml up to about 10 mg/ml. In one embodiment, the total amount of terpene present in the formulation is from about 3.0 mg/ml up to about 10 mg/ml. In one embodiment, the total amount of terpene present in the formulation is from about 4.0 mg/ml up to about 10 mg/ml. In one embodiment, the total amount of terpene present in the formulation is from about 5.0 mg/ml up to about 10 mg/ml.

In one embodiment, the total amount of terpene present in the formulation is from about 0.1 mg/ml up to about 9.0 mg/ml. In one embodiment, the total amount of terpene present in the formulation is from about 0.1 mg/ml up to about 8.0 mg/ml. In one embodiment, the total amount of terpene present in the formulation is from about 0.1 mg/ml up to about 7.0 mg/ml. In one embodiment, the total amount of terpene present in the formulation is from about 0.1 mg/ml up to about 6.0 mg/ml. In one embodiment, the total amount of terpene present in the formulation is from about 0.1 mg/ml up to about 5.0 mg/ml. In one embodiment, the total amount of terpene present in the formulation is from about 0.1 mg/ml up to about 1 mg/ml. In one embodiment, the total amount of terpene present in the formulation is from about 0.1 mg/ml up to about 0.9 mg/ml. In one embodiment, the total amount of terpene present in the formulation is from about 0.1 mg/ml up to about 0.8 mg/ml. In one embodiment, the total amount of terpene present in the formulation is from about 0.1 mg/ml up to about 0.7 mg/ml. In one embodiment, the total amount of terpene present in the formulation is from about 0.1 mg/ml up to about 0.6 mg/ml. In one embodiment, the total amount of terpene present in the formulation is from about 0.1 mg/ml up to about 0.5 mg/ml.

For the avoidance of doubt, combinations of the above end points are explicitly envisaged by the present disclosure. This applies to any of the ranges disclosed herein.

The one or more other functional constituents may comprise one or more of coloring agents, preservatives, binders and/or fillers.

In one embodiment, the formulation may also comprise one or more organic or inorganic acids and their corresponding salts. In one embodiment, the organic acid is a carboxylic acid and the inorganic acid is a phosphoric acid. In one embodiment, the carboxylic acid may be any suitable carboxylic acid, such as a mono-carboxylic acid. In one embodiment, the one or more organic or inorganic acids and their corresponding salts are selected from the group consisting of acetic acid, formic acid, benzoic acid, levulinic acid, succinic acid, oleic acid, sorbic acid, propionic acid, phenylacetic acid, and mixtures thereof.

In one embodiment, the formulation has a pH of less than about 7.5. In this regard, the present inventors have found that when preparing a formulation comprising a cannabinoid, such as cannabidiol, it may be desirable to have a low pH in order to prevent the conversion of cannabidiol to other cannabinoids and to stabilise the formulation. Moreover, a low pH may also be desirable to prevent the formation of CBDHQ. In one embodiment, the formulation has a pH of less than about 7.4. In one embodiment, the formulation has a pH of less than about 7.3. In one embodiment, the formulation has a pH of less than about 7.2. In one embodiment, the formulation has a pH of less than about 7.1. In one embodiment, the formulation has a pH of less than about 7.0. In one embodiment, the formulation has a pH of less than about 6.5. In one embodiment, the formulation has a pH of from about 5.5 to 7.5. In one embodiment, the formulation has a pH of from about 5.5 to 7.4. In one embodiment, the formulation has a pH of from about 5.5 to 7.3. In one embodiment, the formulation has a pH of from about 5.5 to 7.2. In one embodiment, the formulation has a pH of from about 5.5 to 7.1. In one embodiment, the formulation has a pH of from about 5.5 to 7. In one embodiment, the formulation has a pH of from about 6 to 7.5. In one embodiment, the formulation has a pH of from about 6 to 7.4. In one embodiment, the formulation has a pH of from about 6 to 7.3. In one embodiment, the formulation has a pH of from about 6 to 7.2. In one embodiment, the formulation has a pH of from about 6 to 7.1. In one embodiment, the formulation has a pH of from about 6 to 7. In one embodiment, the formulation has a pH of from about 6.5 to 7. In one embodiment, the formulation has a pH of from about 6 to 6.5. In one embodiment, the formulation has a pH of about 6. In one embodiment, the formulation has a pH of about 6.5. In one embodiment, the formulation has a pH of about 7. In one embodiment, the formulation has a pH of about 7.5.

In one embodiment, the one or more stabilizing components are antioxidants selected from ascorbic acid and sodium ascorbate, wherein ascorbic acid is present in an amount of from 1000 to 1750 ppm and sodium ascorbate is present in an amount of from 250 to 1000 ppm and the formulation has a pH of from about 5.5 to 7.5. In one embodiment, the one or more stabilizing components are antioxidants selected from ascorbic acid and sodium ascorbate, wherein ascorbic acid is present in an amount of from 1250 to 1750 ppm and sodium ascorbate is present in an amount of from 250 to 750 ppm and the formulation has a pH of from about 5.5 to 7.5. In one embodiment, the one or more stabilizing components are antioxidants selected from ascorbic acid and sodium ascorbate, wherein ascorbic acid is present in an amount of from 1000 to 1750 ppm and sodium ascorbate is present in an amount of from 250 to 1000 ppm and the formulation has a pH of from about 6 to 7. In one embodiment, the one or more stabilizing components are antioxidants selected from ascorbic acid and sodium ascorbate, wherein ascorbic acid is present in an amount of from 1250 to 1750 ppm and sodium ascorbate is present in an amount of from 250 to 750 ppm and the formulation has a pH of from about 6 to 7.

In one embodiment, the one or more cannabinoids is CBD or a pharmaceutically acceptable salt thereof, the one or more stabilizing components are antioxidants selected from ascorbic acid and sodium ascorbate, wherein ascorbic acid is present in an amount of from 1000 to 1750 ppm and sodium ascorbate is present in an amount of from 250 to 1000 ppm, and the formulation has a pH of from about 5.5 to 7.5. In one embodiment, the one or more cannabinoids is CBD or a pharmaceutically acceptable salt thereof, the one or more stabilizing components are antioxidants selected from ascorbic acid and sodium ascorbate, wherein ascorbic acid is present in an amount of from 1250 to 1750 ppm and sodium ascorbate is present in an amount of from 250 to 750 ppm, and the formulation has a pH of from about 5.5 to 7.5. In one embodiment, the one or more cannabinoids is CBD or a pharmaceutically acceptable salt thereof, the one or more stabilizing components are antioxidants selected from ascorbic acid and sodium ascorbate, wherein ascorbic acid is present in an amount of from 1000 to 1750 ppm and sodium ascorbate is present in an amount of from 250 to 1000 ppm, and the formulation has a pH of from about 6 to 7. In one embodiment, the one or more cannabinoids is CBD or a pharmaceutically acceptable salt thereof, the one or more stabilizing components are antioxidants selected from ascorbic acid and sodium ascorbate, wherein ascorbic acid is present in an amount of from 1250 to 1750 ppm and sodium ascorbate is present in an amount of from 250 to 750 ppm, and the formulation has a pH of from about 6 to 7.

For the avoidance of doubt, the pH of the formulation applies to all formulations described herein, including those comprising further constituents, i.e. flavors and/or terpenes.

The pH of the formulation can be measured as is common in the art. For example, by using the following protocol:

### Instrument and Reagents

Fisher Scientific Accumet® XL200 and SN #2864832 glass mercury free testing probe
Pure grade ethanol (200 proof)
Orion™ pH calibration standards (pH 4, 6, 10.01)
Falcon™ 15 ml conical plastic test tubes
KCl pH meter storage solution

### pH Meter Calibration Procedure

Calibrate the pH meter using pH calibration standards of pH 4, 6, and 10.01.
Ensure that the calibration is within acceptable slope limits of the meter.
If slope readings fail rerun all three calibration standards.

### Testing/Data Recording Procedure

Provide 0.5 grams of sample into a 15 ml Falcon™ test tube and add 3 grams of pure grade ethanol.
Shake the solution for 5 minutes to ensure homogeneity.
Insert pH testing probe into the test tube and wait for the pH to stabilize.
Record the pH reading.
Rinse the probe with ethanol and water, and repeat the test 4 times.
Record each value to provide an average pH and a % relative standard deviation (RSD).

A calibration slope is the conversion that a pH meter can use to convert the electrode signal in mV to pH. The pH meter determines the calibration slope by measuring the difference in the mV reading of two standardized buffer solutions and divides it by the difference in pH of the standard buffer solutions. Acceptable slope limits of the Fisher Scientific Accumet® XL200 meter are 90% and above of the calibration slope. Readings below 90% of a calibration slope is considered out of specification for calibration.

In some embodiments, the formulation has a turbidity of about 10 NTU or less.

In this regard, the present inventors have found that when preparing a formulation comprising a cannabinoid, such as cannabidiol, it is desirable to ensure that the turbidity of the formulation is 10 NTU or less. When the turbidity of the formulation is above this range, it is a sign that one or more of the constituents of the formulation is not present in the formulation in a stable manner. This could impact the use of the formulation in a number of ways. For example, the user may perceive the lack of stability and form an opinion that the formulation is of inferior quality. Alternatively or additionally, such instability may lead to inefficient transfer of one or more constituents from the formulation to the aerosol. Likewise, such instability may lead to the formulation causing suboptimal performance of any system or device using the formulation. The present inventors have found that issues of stability may be particularly pronounced when the formulation comprises a cannabinoid and have thus found that ensuring the formulation has a turbidity of 10 NTU or less is important.

In some embodiments, the turbidity of the formulation is about 10 NTU or less. In some embodiments, the turbidity of the formulation is about 9 NTU or less. In some embodiments, the turbidity of the formulation is about 8 NTU or less. In some embodiments, the turbidity of the formulation is about 7 NTU or less. In some embodiments, the turbidity of the formulation is about 6 NTU or less. In some embodiments, the turbidity of the formulation is about 5 NTU or less. In some embodiments, the turbidity of the formulation is about 4 NTU or less. In some embodiments, the turbidity of the formulation is about 3 NTU or less. In some embodiments, the turbidity of the formulation is about 2 NTU or less. In some embodiments, the turbidity of the formulation is about 1.5 NTU or less. In some embodiments, the turbidity of the formulation is about 1 NTU or less. In some embodiments, the turbidity of the formulation is about 0.9 NTU or less.

In some embodiments, the turbidity of the formulation is about 0.8 NTU or less. In some embodiments, the turbidity of the formulation is about 0.7 NTU or less. In some embodiments, the turbidity of the formulation is about 0.6 NTU or less. In some embodiments, the turbidity of the formulation is about 0.5 NTU or less. In some embodiments, the turbidity of the formulation is about 0.4 NTU or less. In some embodiments, the turbidity of the formulation is about 0.3 NTU or less. In some embodiments, the turbidity of the formulation is about 0.2 NTU or less.

In some embodiments, the turbidity of the formulation is from about 0.1 NTU to about 1 NTU. In some embodiments, the turbidity of the formulation is from about 0.2 NTU to about 1 NTU. In some embodiments, the turbidity of the formulation is from about 0.3 NTU to about 1 NTU. In some embodiments, the turbidity of the formulation is from about 0.4 NTU to about 1 NTU. In some embodiments, the turbidity of the formulation is from about 0.5 NTU to about 1 NTU. In some embodiments, the turbidity of the formulation is from about 0.1 NTU to about 0.9 NTU. In some embodiments, the turbidity of the formulation is from about 0.1 NTU to about 0.8 NTU. In some embodiments, the turbidity of the formulation is from about 0.1 NTU to about 0.7 NTU. In some embodiments, the turbidity of the formulation is from about 0.1 NTU to about 0.6 NTU. In some embodiments, the turbidity of the formulation is from about 0.1 NTU to about 0.5 NTU. The turbidity of the formulation can be measured as is common in the art. For example, by using a TL2310 ISO Turbidimeter from Hach, Colorado, 80539-0389, United States.

In some embodiments, an acceptable turbidity is achieved without the use of functional constituents which influence the stability of the formulation. For example, it may be possible to decrease the turbidity of a liquid system by introducing surface active constituents which serve to improve the emulsification/dispersion of one or more of the constituents. However, it may not be desirable to include such functional constituents due to user acceptability. Therefore, in some embodiments, the formulation does not comprise a surface active constituent. Examples of surface active constituents include medium chain triglycerides (MCT) and tocopherol acetate.

In some embodiments, an acceptable turbidity is achieved without the use of any/significant amounts of water. In this regard, whilst water may otherwise assist in the preparation of formulations since water containing materials may have a lower viscosity and therefore may be transferred more easily to an aerosol generating component, it has been found in the context of the present disclosure that water can negatively influence the stability of the formulation containing at least one cannabinoid.

In some embodiments, the formulation comprises less than 12% w/w water. In some embodiments, the formulation comprises less than 11% w/w water. In some embodiments, the formulation comprises less than 10% w/w water. In some embodiments, the formulation comprises less than 5% w/w water. In some embodiments, the formulation comprises less than 1% w/w water. In some embodiments, the formulation comprises less than 0.5% w/w water. In some embodiments, the formulation comprises substantially no water.

In particular, it has been found that for certain formulations comprising a cannabinoid, such as cannabidiol, if the formulation comprises water in amounts of about 12% w/w, the cannabinoid is rendered unstable.

In one embodiment, the formulations described herein are storage stable.

In this regard, the present inventors have found that the formulations maintain a high degree of stability, even when exposed to air and/or light, and/or variations in temperature.

In a further aspect there is provided a formulation as defined herein, wherein the formulation is storage stable when the content of one or more specific cannabinoids is at least 80% of the initial content of the one or more specific cannabinoids based on a mg/ml basis of the formulation after 4 weeks at 40° C. and 75% Relative Humidity.

In one embodiment the formulations described are formulated such that the content of one or more specific cannabinoids, such as cannabidiol (CBD), is at least 80% of the initial content of the one or more cannabinoids based on a mg/ml basis of the formulation after 4 weeks at 40° C. and 75% Relative Humidity.

In one embodiment, the content of one or more specific cannabinoids is at least 85% of the initial content of the one or more cannabinoids based on a mg/ml basis. In one embodiment, the content of one or more specific cannabinoids is at least 90% of the initial content of the one or more cannabinoids based on a mg/ml basis. In one embodiment, the content of one or more specific cannabinoids is at least 95% of the initial content of the one or more cannabinoids based on a mg/ml basis. In one embodiment, the content of one or more specific cannabinoids is at least 97% of the initial content of the one or more cannabinoids based on a mg/ml basis.

The formulations described herein may be produced by combining each of the one or more cannabinoids, one or more stabilizing components and one or more carrier constituents so as to form the formulation, wherein the one or more stabilizing components and one or more carrier constituents are combined to form a first mixture, and then the one or more cannabinoids is added to the first mixture to produce the formulation.

The one or more stabilizing components and one or more carrier constituents may be combined in a container to form the first mixture. The first mixture may be subjected to stirring. The stirring may take place at between 200 to 600 rpm, and for between 12 to 48 hours. The one or more carrier constituents used to form the first mixture may be propylene glycol.

One or more cannabinoids are then added to the first mixture. The resulting mixture may be subjected to stirring. The stirring may take place at between 200 to 600 rpm, and for between 2 to 8 hours.

The resulting mixture may optionally be filtered, for example using a 0.2 μm.

To this mixture may be added one or more additional carrier constituents. These carrier constituents may be the same and/or different as those added to create the first mixture. For example, propylene glycol and/or glycerol may be added.

Optionally one or more olfactory active components as defined herein may be added. Typically, such components are added after the additional carrier constituents have been added. The formulations may then be subject to further packaging.

In one embodiment the formulations described herein are vaporizable formulations. For example, the formulations described herein are vaporizable formulations that are suitable for use with an aerosol provision system, such as an e-cigarette. In one embodiment the formulations described herein are vaporizable liquids.

In one embodiment the formulations described herein are packaged formulations, wherein the packaged formulations are impermeable to air. In this regard, formulations can be prepared with predefined amounts of one or more cannabinoids that maintain a high degree of stability. As defined herein, impermeable to air implies that the packaged formulations are closed or sealed to provide substantial air impermeability.

In one embodiment the packaged formulations are packaged in a container that is substantially impermeable to air and/or light (including UV light), which can be used with an aerosol provision system. The container may correspond to a store comprising a formulation as defined herein. In this regard, the stability of the packaged formulations may be maintained during use.

In one embodiment the packaged formulations are packaged in a container, which comprise the formulations described herein and a gas, such as air, CO₂, N₂ or a noble gas. In this regard, the volume of said gas in the container is referred to as the headspace. So that the stability of the packaged formulation can be maintained, it is preferable to reduce the volume of headspace in the container. In one embodiment there is a method of preparing a packaged formulation, comprising packaging a formulation as described herein in a container that is substantially impermeable to air and/or light (including UV light), wherein the container further comprises a volume of gas no greater than 20% of the total volume of the container. In one embodiment the volume of gas is no greater than 15% of the total volume of the container. In one embodiment the volume of gas is no greater than 10% of the total volume of the container. In one embodiment the volume of gas is no greater than 5% of the total volume of the container. In one embodiment the volume of gas is no greater than 1% of the total volume of the container.

In one embodiment there is a container comprising a formulation as described herein, wherein the container further comprises a volume of gas no greater than 20% of the total volume of the container, wherein the gas may be air, CO₂, N₂ or a noble gas. In one embodiment the volume of gas is no greater than 15% of the total volume of the container. In one embodiment the volume of gas is no greater than 10% of the total volume of the container. In one embodiment the volume of gas is no greater than 5% of the total volume of the container. In one embodiment the volume of gas is no greater than 1% of the total volume of the container.

The noble gas referred to herein may be argon.

In one embodiment, the packaged formulations and containers described herein are sealed in one or more blister packs that are substantially impermeable to air and light (such as ultra violet light). In this regard, the stability of the formulations and packaged formulations may be maintained during storage.

In one aspect the formulations described herein are aerosolisable materials.

In a further aspect there is provided an article comprising the formulation as defined herein.

The article may be a container, such as a bottle, or may be a component for use with an aerosol provision device.

For example, the article may comprise an area (store) for receiving the formulation defined herein, an aerosol generating component, an aerosol generating area, and/or a mouthpiece.

In some embodiments, there is provided an article for use with an aerosol provision system, the article comprising a store comprising a formulation as defined herein, an aerosol generating component (such as a heater), an aerosol generating area, a transport element, and a mouthpiece.

Formulation may be transferred from the store for receiving a formulation to the aerosol generating component via a transport element, such as a wick, pump or the like. The skilled person is able to select suitable transport elements depending on the type of formulation that is to be transported and the rate at which it must be supplied. Particular mention may be made of transport elements, such as wicks, formed from fibrous materials, foamed materials, sintered materials, woven and non-woven materials.

An airflow pathway typically extends through the article (optionally via the device) to an outlet. The pathway is oriented such that generated aerosol is entrained in the airflow such that it can be delivered to the outlet for inhalation by a user.

In one embodiment, the aerosol generating component is a heater.

Typically, the area for receiving a formulation will allow for the article to be refilled with formulation as the formulation is depleted during use.

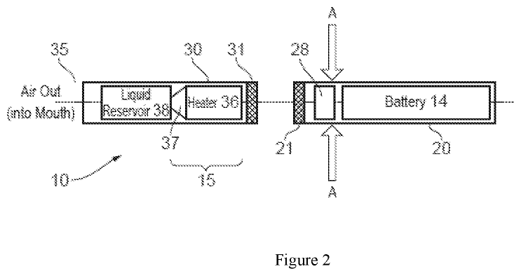

FIG. 2 is a highly schematic diagram (not to scale) of an example aerosol provision system, such as an e-cigarette 10, to which embodiments are applicable. The e-cigarette has a generally cylindrical shape, extending along a longitudinal axis indicated by a dashed line (although aspects of the invention are applicable to e-cigarettes configured in other shapes and arrangements), and comprises two main components, namely an aerosol provision device 20 and an article 30.

The article 30 includes a store for the formulation (source liquid) 38 containing a formulation (source liquid) from which an aerosol is to be generated. The article 30 further comprises an aerosol generating component (heating element or heater) 36 for heating the formulation to generate the aerosol. A transport element or wicking element or wick 37 is provided to deliver the formulation from the store 38 to the heating element 36. A part or parts of the wick 37 are in fluid communication with the formulation in the store 38 and by a wicking or capillary action the formulation is drawn along or through the wick 37 to a part or parts of the wick 37 which are in contact with the heater 36.

Vaporization of the formulation occurs at the interface between the wick 37 and the heater 36 by the provision of heat energy to the formulation to cause evaporation, thus generating the aerosol. The formulation, the wick 37 and the heater 36 may be collectively referred to as an aerosol or vapour source. The wick 37 and the heater 36 may be collectively referred to as a vaporizer or an atomiser 15.

Typically a single wick will be present, but it is envisaged that more than one wick could be present, for example, two, three, four or five wicks.

As described above, the wick may be formed a sintered material. The sintered material may comprise sintered ceramic, sintered metal fibres/powders, or a combination of the two. The (or at least one of/all of the) sintered wick(s) may have deposited thereon/embedded therein an electrically resistive heater. Such a heater may be formed from heat conducting alloys such as NiCr alloys. Alternatively, the sintered material may have such electrical properties such that when a current is passed there through, it is heated. Thus, the aerosol generating component and the wick may be considered to be integrated. In some embodiments, the aerosol generating component and the wick are formed from the same material and form a single component.

In some embodiments, the wick is formed from a sintered metal material and is generally in the form of a planar sheet. Thus, the wick element may have a substantially thin flat shape. For example it may be considered as a sheet, layer, film, substrate or the like. By this it is meant that a thickness of the wick is less or very much less than at least one of the length and the width of the wick. Thus, the wick thickness (its smallest dimension) is less or very much less than the longest dimension.

The wick may be made of a homogenous, granular, fibrous or flocculent sintered metal(s) so as to form said capillary structure. Wick elements can be made from a conductive material which is a nonwoven sintered porous web structure comprising metal fibres, such as fibres of stainless steel. For example, the stainless steel may be AISI (American Iron and Steel Institute) 316L (corresponding to European standard 1.4404). The material's weight may be in the range of 100-300 g/m².

Where the wick is generally planar, the thickness of the wick may be in the range of 75-250 μm. A typical fibre diameter may be about 12 μm, and a typical mean pore size (size of the voids between the fibres) may be about 32 μm. An example of a material of this type is Bekipor® ST porous metal fibre media manufactured by NV Bekaert SA, Belgium, being a range of porous nonwoven fibre matrix materials made by sintering stainless steel fibres.

Note also that while the material is described as planar, this refers to the relative dimensions of the sheet material and the wick (a thickness many times smaller than the length and/or width) but does not necessarily indicate flatness, in particular of the final wick made from the material. A wick may be flat but might alternatively be formed from sheet material into a non-flat shape such as curved, rippled, corrugated, ridged, formed into a tube or otherwise made concave and/or convex.

The wick element may have various properties. It is formed from a porous material to enable the required wicking or capillary effect for drawing source liquid through it from an store for the formulation (where the wick meets the formulation at a store contact site) to the vaporisation interface. Porosity is typically provided by a plurality of interconnected or partially interconnected pores (holes or interstices) throughout the formulation, and open to the outer surface of the formulation. Any level of porosity may be employed depending on the formulation, the size of the pores and the required rate of wicking. For example a porosity of between 30% and 85% might be selected, such as between 40% and 70%, between 50% and 80%, between 35% and 75% or between 40% and 75%. This might be an average porosity value for the whole wick element, since porosity may or may not be uniform across the wick. For example, pore size at the store contact site might be different from pore size nearer to the heater.

It is useful for the wick to have sufficient rigidity to support itself in a required within the article. For example, it may be mounted at or near one or two edges and be required to maintain its position substantially without flexing, bending or sagging.

As an example, porous sintered ceramic is a useful material to use as the wick element. Any ceramic with appropriate porosity may be used. If porous ceramic is chosen as the porous wick material, this is available as a powder which can be formed into a solid by sintering (heating to cause coalescence, possibly under applied pressure). Sintering then solidifies the ceramic to create the porous wick.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section.

During operation of the device, the controller will determine that a user has initiated a request for the generation of an aerosol. This could be done via a button on the device which sends a signal to the controller that the aerosol generator should be powered. Alternatively, a sensor located in or proximal to the airflow pathway could detect airflow through the airflow pathway and convey this detection to the controller. A sensor may also be present in addition to the presence of a button, as the sensor may be used to determine certain usage characteristics, such as airflow, timing of aerosol generation etc.

For example, in use, when the heater 36 receives power from the battery 14, as controlled by the circuit board 28 possibly in response to pressure changes detected by an air pressure sensor (not shown), the heater 36 vaporizes the formulation delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 2) that connects the aerosol source to the mouthpiece opening as a user inhales on the mouthpiece.

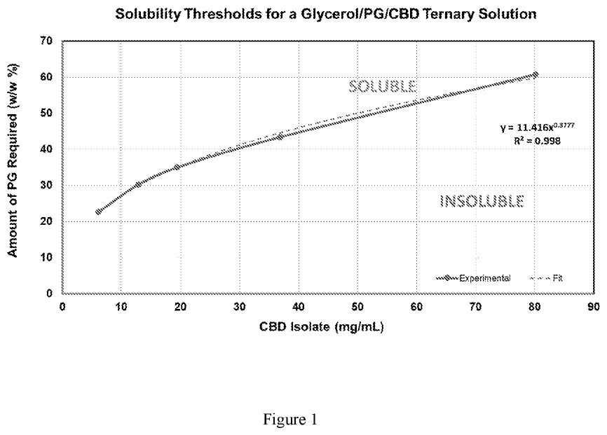

In this particular example, the device 20 and article 30 are detachable from one another by separation in a direction parallel to the longitudinal axis, as shown in FIG. 1, but are joined together when the system 10 is in use by cooperating engagement elements 21, 31 (for example, a screw, magnetic or bayonet fitting) to provide mechanical and electrical connectivity between the device 20 and the article 30, in particular connecting the heater 36 to the battery 14. The battery may be charged as is known to one skilled in the art.

In some embodiments, the article comprises/forms a sealed container. For example, the sealed container may be hermetically sealed. The present inventors have found that inclusion of the formulation in a sealed article assists in preventing water ingress into the system, which can prevent the cannabinoid from precipitating. The hermetically sealed container may comprise a blister pack with one or more hermetically sealed compartments for storage of one or more articles comprising the formulation described herein.

In some embodiments, the article comprises a housing within which the formulation is contained. The housing may be transparent such that the formulation can be viewed from outside of the housing. It may also be that the housing has a degree of opacity such that the passage of light through the housing is limited. This can be important so as to prevent light (such as ultra violet light) from entering the housing and compromising the stability of the formulation. In this regard, the present inventors have considered that cannabinoids may be particularly susceptible to such light destabilization. In some embodiments, the housing is formed from a material which inhibits the passage of ultra violet light there through. In some embodiments, it may be that the sealed container mentioned above is formed from a material which has a degree of opacity such that the passage of light through the sealed container is limited. Further, the sealed container mentioned above may be formed from a material which inhibits/prevents the passage of ultra violet light there through. This may be in addition to said sealed container being hermetically sealed and/or comprising a blister pack with one or more hermetically sealed compartments for storage of one or more articles comprising the formulation described herein.

In a further aspect there is provided an aerosol provision system comprising an aerosol provision device and an article as defined herein.

In a further aspect, there is provided a method for producing an aerosol comprising generating an aerosol from a formulation as defined herein.

### EXAMPLES

### Method for Preparing CBD Formulations

Stock formulation is prepared by combining propylene glycol (PG) and stabilizing component in a container and stirring at 200-600 rpm for 12-48 hours to allow dissolution.
CBD is then added to the PG/stabilizing component solution and stirred at 200-600 rpm for 2-8 hours to allow dissolution.
The solution is filtered through 0.2 μm filter to remove any particulates.
Samples of the formulation are taken to determine specific gravity and refractive index (SG/RI), wt % CBD and presence of other cannabinoids and derivatives (e.g. CBDHQ).
The required amount of stock formulation is added to a container based upon on the desired CBD content of the final CBD formulation and the % CBD in stock formulation.
PG is added to dilute the stock formulation.
Flavor compounds and other additives may be added to the formulation at this point while stirring.
Glycerol may then be added to the formulation and stirred at 200-600 rpm for 15-30 minutes to homogenize the components. (See FIG. 1 for information regarding solubility of ternary CBD/PG/Glycerol formulations).
Samples of the formulation are taken to determine specific gravity and refractive index (SG/RI), wt % CBD and presence of other cannabinoids and derivatives (e.g. CBDHQ).
Formulation is dispensed into product containers and argon layer may be applied prior to sealing and storing at 2-8° C.

### Example 1

Formation of CBDHQ was assessed in CBD formulations under accelerated test conditions. The samples comprised 70% w/w propylene glycol and 30% w/w glycerol, based on the total amount of propylene glycol and glycerol in the formulation, 60 mg/ml CBD and one or more stabilizing components provided in 0.22 M equivalents relative to CBD. All samples were unflavoured and identical, with the exception of the specified stabilizing component(s). A control sample comprising no stabilizing components was used for comparative analysis. Samples were stored for 14 days at 40° C. in dark storage (no light).

TABLE 1

| Sample | Stabilizing Component | CBDHQ (ppm) |
|---|---|---|
| 1 | Control | 90 |
| 2 | Ascorbic Acid | 3 |
| 3 | Ethyl Maltol | 38 |
| 4 | Pyruvic Acid | 25 |
| 5 | Lactic | 73 |

Reduced levels of CBDHQ were observed in all samples comprising stabilizing components.

### Example 2

Formation of CBDHQ in CBD formulations was assessed under accelerated test conditions. The samples comprised 70% w/w propylene glycol and 30% w/w glycerol, based on the total amount of propylene glycol and glycerol in the formulation, 60 mg/ml CBD and either ascorbic acid or ethyl maltol in varying concentrations. All samples were comparable, with the exception that the concentration of ascorbic acid or ethyl maltol differs. A control sample comprising no stabilizing components was used for comparative analysis. Samples were stored for 21 days at 40° C. in dark storage (no light).

TABLE 2

| Sample | Stabilizing Component | CBDHQ (ppm) |
|---|---|---|
| 1 | Control | 65 |
| 2 | Ascorbic Acid (500 ppm) | 1 |
| 3 | Ascorbic Acid (300 ppm) | 3 |
| 4 | Ascorbic Acid (100 ppm) | 42 |
| 5 | Ethyl Maltol (1000 ppm) | 30 |
| 6 | Ethyl Maltol (300 ppm) | 43 |

Reduced levels of CBDHQ were observed in all samples.

### Example 3

Formulations comprising CBD were analysed for the formation of CBDHQ and CBN under ambient test conditions. The formulations were prepared in accordance with the above method and comprised 70% w/w propylene glycol and 30% w/w glycerol, based on the total amount of propylene glycol and glycerol in the formulation, 60 mg/ml CBD isolate with ascorbic acid (“AA”) and/or sodium ascorbate (“Asb”) in varying concentrations (ppm). A control sample comprising no stabilizing components was used for comparative analysis. The CBDHQ and CBN content

TABLE 3
CBDHQ Formation

| | | | | CBDHQ Time (Days) | | | | |
|---|---|---|---|---|---|---|---|---|
| Sample | Stabilizer Cmp | | pH | 0 | 28 | 56 | 84 | 105 |
| 1 Control | None | | 6.67 | 1.70 | 18.20 | 274.00 | 486.00 | 784.60 |
| 2 CBD | 250 AA/250 Asb | | 6.98 | 0.20 | 2.20 | 6.60 | 35.00 | 115.48 |
| 3 CBD | 500 | Asb | 7.62 | 0.10 | 2.10 | 12.50 | 64.00 | 232.43 |
| 4 CBD | 500 | AA | 6.12 | 0.20 | 0.40 | 14.50 | 21.00 | 138.61 |

TABLE 4
CBN Formation

| | | | | CBN Time (Days) | | | | |
|---|---|---|---|---|---|---|---|---|
| Sample | Stabilizer Cmp | | pH | 0 | 28 | 56 | 84 | 105 |
| 1 Control | None | | 6.67 | 0.40 | 0.35 | 0.35 | 0.42 | 0.69 |
| 2 CBD | 250 AA/250 Asb | | 6.98 | 0.33 | 0.33 | 0.31 | 0.40 | 0.50 |
| 3 CBD | 500 | Asb | 7.62 | 0.40 | 0.30 | 0.33 | 0.40 | 0.65 |
| 4 CBD | 500 | AA | 6.12 | 0.37 | 0.27 | 0.32 | 0.41 | 0.50 |

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A formulation comprising: one or more cannabinoids, one or more stabilizing component and one or more carrier constituents, wherein the total amount of the one or more carrier constituents is 50% w/w or more based on the total weight of the formulation, wherein said formulation has a pH of less than about 7.5.

2. The formulation according to claim 1, wherein the cannabinoids are selected from cannabigerol (CBG), cannabichromene (CBC), cannabidiol (CBD), Tetrahydrocannabinol (THC), including its isomers Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

3. The formulation according to claim 2, wherein the cannabinoids are selected from cannabigerol (CBG), cannabichromene (CBC), cannabidiol (CBD), Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC) and cannabinol (CBN).

4. The formulation according to claim 2, wherein the cannabinoids are selected from cannabidiol (CBD), Δ⁹-tetrahydrocannabinol (Δ⁹-THC), and cannabinol (CBN).

5. The formulation according to claim 2, wherein the cannabinoids comprise cannabidiol (CBD).

6. The formulation according to claim 1, wherein the cannabinoid is cannabidiol.

7. The formulation according to claim 1, wherein the cannabinoids comprise cannabidiol (CBD) and one or more cannabinoids selected from cannabigerol (CBG), cannabichromene (CBC), cannabidiol (CBD), Tetrahydrocannabinol (THC), including its isomers Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

8. The formulation according to claim 7, wherein the cannabinoids comprise cannabidiol (CBD) and one or more cannabinoids selected from cannabigerol (CBG), cannabichromene (CBC), Δ^{6a,10a}-Tetrahydrocannabinol (Δ^{6a,10a}-THC), Δ^{6a(7)}-Tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC) and cannabinol (CBN).

9. The formulation according to claim 7, wherein the cannabinoids comprise cannabidiol (CBD) and one or more cannabinoids selected from Δ⁹-tetrahydrocannabinol (Δ⁹-THC) and cannabinol (CBN).

10. The formulation according to claim 1, wherein the one or more stabilizing components are selected from antioxidants, pH modulators, chelators and radical scavengers, and combinations thereof.

11. The formulation according to claim 10, wherein the one or more stabilizing components are one or more antioxidants and one or more chelators.

12. The formulation according to claim 10, wherein the one or more antioxidants are selected from the enediol class of compounds.

13. The formulation according to claim 1, wherein the one or more stabilizing components are selected from the group consisting of ascorbic acid, sodium ascorbate, ethyl maltol, thymol, maltol, pyruvic acid, lactic acid, carvacrol, alpha-keto glutaric acid, alpha-keto glutarate salt, triethyl citrate, ethyl vanillate, quercetin, sucrose acetate isobutyrate, retinol, cholecalciferol, vitamin K-hydroquinone, citric acid, tartaric acid, ferulic acid, courmaric acid, propyl gallate, gallic acid, alpha lipoic acid, ascorbyl palmitate, lutein, lycopene, resveratrol, rutin, catechin, carnosol, rosmarinic acid, lipoic acid, α-resorcylic, pyrogallol, malvidin, theaflavin, apigenin, eriodictyol, glycitein, chrysoeriol, kaempferol, luteolin, vitexin, isovitexin, orientin, cannflavin A, cannflavin B, cannflavin C, delphinidin, pelargonidin, epicatechin, myricetin, chrysin, naringenin, α-terpineol, nerol, geranyl acetate, fenchol, propyl gallate, tert-butylhydroquinone, carvone and combinations thereof.

14. The formulation according to claim 13, wherein the one or more stabilizing components are selected from one or more of ascorbic acid and sodium ascorbate.

15. The formulation according to claim 14, wherein the stabilizing components are ascorbic acid and sodium ascorbate.

16. The formulation according to claim 1, wherein the one or more stabilizing components are each present in an amount of at least 500 ppm.

17. The formulation according to claim 1, wherein the one or more stabilizing components are each present in an amount of at least 1000 ppm.

18. The formulation according to claim 17, wherein the one or more stabilizing components are each present in an amount of at least 1500 ppm.

19. The formulation according to claim 18, wherein the one or more stabilizing components are each present in an amount of at least 2000 ppm.

20. The formulation according to claim 1, wherein the formulation has a pH of from about 6 to about 7.

21. The formulation according to claim 20, wherein the formulation has a pH of from about 6.5 to about 7.

22. The formulation according to claim 20, wherein the formulation has a pH of from about 6 to about 6.5.

23. The formulation according to claim 1, wherein the carrier constituent comprises one or more of glycerol, propylene glycol, triethylene glycol, tetraethylene glycol, 1,3-butylene glycol, erythritol, meso-Erythritol, ethyl laurate, a diethyl suberate, triethylene glycol diacetate, triacetin, a diacetin mixture, benzyl benzoate, benzyl phenyl acetate, tributyrin, lauryl acetate, lauric acid, myristic acid, and propylene carbonate.

24. The formulation according to claim 23, wherein the total amount of carrier constituents is 60% w/w or more based on the total weight of the formulation.

25. The formulation according to claim 23, wherein the total amount of carrier constituents is 70% w/w or more based on the total weight of the formulation.

26. The formulation according to claim 1, wherein the carrier constituent comprises propylene glycol.

27. The formulation according to claim 26, wherein propylene glycol is present in an amount of at least 50% w/w based on the total weight of the formulation.

28. The formulation according to claim 26, wherein propylene glycol is present in an amount of at least 60% w/w based on the total weight of the formulation.

29. The formulation according to claim 26, wherein propylene glycol is present in an amount of at least 70% w/w based on the total weight of the formulation.

30. The formulation according to claim 1, wherein the carrier constituent comprises glycerol.

31. The formulation according to claim 29, wherein glycerol is present in an amount of at least 50% w/w based on the total weight of the formulation.

32. The formulation according to claim 30, wherein glycerol is present in an amount of at least 60% w/w based on the total weight of the formulation.

33. The formulation according to claim 30, wherein glycerol is present in an amount of at least 70% w/w based on the total weight of the formulation.

34. The formulation according to claim 26, wherein both glycerol and propylene glycol are present as carrier constituents.

35. The formulation according to claim 34, wherein the formulation comprises:
60 to 90% w/w propylene glycol and 40 to 10% w/w glycerol based on the total amount of propylene glycol and glycerol in the formulation.

36. The formulation according to claim 34, wherein the formulation comprises 70 to 80% w/w propylene glycol and 30 to 20% w/w glycerol based on the total amount of propylene glycol and glycerol in the formulation.

37. The formulation according to claim 34, wherein the formulation comprises about 70% w/w propylene glycol and about 30% w/w glycerol based on the total amount of propylene glycol and glycerol in the formulation.

38. The formulation according to claim 1, wherein at least one cannabinoid is present in the formulation in an amount of 5 mg/ml or more.

39. The formulation according to claim 38, wherein at least one cannabinoid is present in the formulation in an amount of 10 mg/ml or more.

40. The formulation according to claim 38, wherein at least one cannabinoid is present in the formulation in an amount of 30 mg/ml or more.

41. The formulation according to claim 38, wherein at least one cannabinoid is present in the formulation in an amount of 60 mg/ml or more.

42. The formulation according to claim 1, wherein the formulation additionally comprises one or more terpenes selected from pinene (alpha and beta), geraniol, linalool, limonene, eucalyptol, menthone, iso-menthone, piperitone, beta-bourbonene, germacrene and myrcene and mixtures thereof.

43. The formulation according to claim 42, wherein the total amount of terpene present in the formulation is up to about 10 mg/ml.

44. The formulation according to claim 1, wherein the formulation further comprises one or more active constituents in addition to the cannabinoid.

45. The formulation according to claim 44, wherein the one or more active constituents is an olfactory active constituent.

46. The formulation according to claim 1, wherein the formulation takes the form of a liquid at about 25° C.

47. The formulation according to claim 1, wherein the content of one or more specific cannabinoids is at least 80% of the initial content of the one or more specific cannabinoids based on a mg/ml basis of the formulation after 4 weeks at 40° C. and 75% Relative Humidity.

48. A packaged formulation comprising a formulation according claim 1 and wherein the packaged formulation is impermeable to air.

49. A method of producing a formulation according to claim 1, wherein the method comprises combining each of the one or more cannabinoids, one or more stabilizing components and one or more carrier constituents so as to form the formulation, wherein the one or more stabilizing components and one or more carrier constituents are combined to form a first mixture, and then the one or more cannabinoids is added to the first mixture to produce the formulation.

50. A method of preparing a packaged formulation, wherein the method comprises packaging a formulation according to claim 1 in a container, wherein the container further comprises a volume of gas no greater than 20% of the total volume of the container.

51. A container comprising a formulation according to claim 1, wherein the container further comprises a volume of gas no greater than 20% of the total volume of the container.

52. A container according to claim 51, wherein the gas is argon.
